(19) United States
(12) Patent Application Publication
(10) Pub. No.: US 2004/0241103 A1
(43) Pub. Date: Dec. 2, 2004

(54) Pharmaceutical aerosol compositions

(75) Inventors: Yvonne M. Pritchard, Nottingham (GB); Kanwaljit S. Claire, Leicester (GB)

Correspondence Address: FINNEGAN, HENDERSON, FARABOW, GARRETT & DUNNER, LLP, 1300 I STREET, NW, WASHINGTON, DC, 20005, US

(73) Assignee: 3M Innovative Properties Company

(21) Appl. No.: 10/886,688

(22) Filed: Jul. 9, 2004

Related U.S. Application Data
(63) Continuation of application No. 10/221,261, filed on Sep. 10, 2002, now abandoned.
Application No. 10/221,261 is a 371 of international application No. PCT/GB98/03488, filed on Nov. 20, 1998.

(51) Int. Cl. A61L 9/04; A61K 9/14

(52) U.S. Cl. 424/46; 514/171; 514/651

(57) ABSTRACT

A medicinal aerosol formulation comprising propellant particles of a first drug suspended in propellant and a second drug completely dissolved in the aerosol formulation.

### FIELD OF THE INVENTION

This invention relates to pharmaceutical aerosol compositions and in particular to pharmaceutical aerosol compositions comprising a plurality of active ingredients.

### BACKGROUND OF THE INVENTION

The use of a self-propelling composition comprising one or more aerosol propellants as a delivery system for medicament-has been known for many years. Such aerosol compositions have been used for topical applications and for delivering of medicaments to the respiratory system of a patient. Since the metered dose inhaler (MDI) was introduced in the mid 1950's, inhalation has become a widely used route for delivery of drugs for the treatment of bronchial maladies and as an alternative to other administration routes for drug delivery for treatment of maladies which are not primarily concerned with the respiratory system.

MDI formulations generally comprise one or more propellants, drug which is dissolved or suspended in the propellent and a surfactant to aid in dissolution or suspension of the drug in propellant, to act as a lubricant for the valve and/or stabilise the formulation. The formulations often comprise a further adjuvant, such as a cosolvent, to solubilise the surfactant and/or drug in the propellant. It is important that the drug be either readily soluble or substantially insoluble in the formulations. Partial dissolution can result in problems with crystal growth and for inhalation therapy particles of from 1 to 10 μm are required for penetration into the bronchioles or pulmonary regions of the lung.

Most of the medicinal aerosol formulations which are available contain a single active ingredient. However, some “compound” preparations containing two or more drugs are known. The following are examples of marketed products containing two or more drugs in suspension formulations. The aerosol formulation commercially available under the trade name Combivent from Boehringer Ingelheim comprises a mixture of ipratropium bromide and salbutamol sulphate.

The aerosol formulation commercially available under the trade name Duovent from Boehringer Ingelheim comprises a mixture of fenoterol hydrobromide and ipratropium bromide.

The aerosol formulation commercially available under the trade name Ventide from Allen & Hanburys Limited comprises a mixture of beclomethasone dipropionate and salbutamol.

The aerosol formulation commercially available under the trade name Aerocrom from Fisons comprises a mixture of disodium cromoglycate and salbutamol sulphate.

The aerosol formulation commercially available under the trade name Aarane from Fisons Arzneimittel GmbH comprises a mixture of disodium cromoglycate and reproterol hydrochloride.

The aerosol formulation commercially available under the trade name Tobispray from Boehringer Ingelheim comprises a mixture of tramazoline hydrochloride and dexamethasone-21-isonicotinate.

The aerosol formulation commercially available under the trade name Duo-Medihaler from 3M UK plc comprises a mixture of isoproterenol hydrochloride and phenylephrine bitartrate.

The aerosol formulation commercially available under the trade name Allergospasmin from Degussa Pharma Gruppe comprises a mixture of disodium cromoglycate and reproterol hydrochloride.

The aerosol formulation commercially available under the trade name Clenil Cemposistum from Chiesi Farmaceutici comprises a mixture of beclomethasone dipropionate and salbutamol.

The aerosol formulation commercially available under the trade name Berodual from Boehringer Ingelheim comprises a mixture of ipratropium bromide and fenoterol hydrobromide.

Other compound preparations are disclosed in U.S. Pat. No. 3,320,125 and WO93/11745.

In all of these known compound preparations the drugs present in a preparation are in the same form i.e., they are both present as suspensions or both in solution.

It has now been found that stable aerosol formulations may be prepared comprising two drugs, one of which is in suspension and the other in solution.

### BRIEF SUMMARY OF THE INVENTION

Therefore, according to the present invention there is provided a medicinal aerosol formulation comprising propellant particles of a first drug suspended in propellant and a second drug completely dissolved in the aerosol formulation. Surprisingly, it has been found that by suitable selection of the components the presence of dissolved drug does not deleteriously affect the stability of the suspended drug particles.

### DESCRIPTION OF PREFERRED EMBODIMENTS

The propellant used in the formulation is preferably a non-CFC propellant e.g. a hydrofluoroalkane (HFA). The most preferred propellants are P134a and P227 which can be used alone or in admixture.

In principle any drug which can be rendered soluble or substantially insoluble in the aerosol formulation may be used in the formulations of the invention. The combination of drugs will be selected for the therapeutic effect. Non-limiting examples of suitable drugs include antiallergics, analgesics, bronchodilators, antihistamines, therapeutic proteins and peptides, antitussives, anginal preparations, antibiotics, anti-inflammatory preparations, hormones, or sulfonamides, such as, for example, a vasoconstrictive amine, an enzyme, an alkaloid or a steroid, and synergistic combinations of these specific examples or drugs which may be employed are: isoproterenol [alpha-(isopropylaminomethyl) protocatechuyl alcohol], phenylephrine, phenylpropanolamine, glucagon, adrenochrome, trypsin, epinephrine, ephedrine, narcotine, codeine, atropine, heparin, morphine, dihydromorphinone, ergotamine, scopolamine, methapyrilene, cyanocobalamin, terbutaline, rimiterol, salbutamol, formoterol, salmeterol, budesonide, isoprenaline, fenoterol, ipratropium bromide, oxitropium bromide, reproterol, fluticasone propionate, flunisolide, triamcinolone acetonide, mometasonefuroate, colchicine, pirbuterol, beclomethasone dipropionate, orciprenaline, fentanyl, and diamorphine. Others are antibiotics, such as neomycin, streptomycin, penicillin, procaine penicillin, tetracycline, chlorotetracycline and hydroxytetracycline; adrenocorticotropic hormone and adrenocortical hormones, such as cortisone, hydrocortisone, hydrocortisone acetate and prednisolone; antiallergy compounds such as cromolyn sodium, nedocromil, protein and peptide molecules such as insulin, pentamidine, calcitonin, amiloride, interferon, LHRH analogues, DNAase, heparin, etc.

The drugs exemplified above may be used as either the free base or as one or more salts known to the art. The choice of free base or salt will be influenced by the physical stability of the drug in the formulation. For example, it has been shown that the free base of salbutamol exhibits a greater dispersion stability than salbutamol sulphate in the aerosol formulations.

The following salts of the drugs mentioned above may be used; acetate, benzenesulphonate, benzoate, bicarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexyiresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate (embonate), panthothenate, phosphate/diphosphate, polygalacturonate, salicylate, stearate, subacetate, succinate, sulphate, tannate, tartrate, triethiodide and xinafoate.

Cationic salts may also be used. Suitable cationic salts include the alkali metals, e.g., sodium and potassium, and ammonium salts and salts of amines known in the art to be pharmaceutically acceptable, e.g., glycine, ethylene diamine, choline, diethanolamine, triethanolamine, octadecylamine, diethylamine; triethylamine, 1-amino-2-propanol-amino-2-(hydroxymethyl)propane-1-3-diol and 1-(3,4-dihydroxyphenyl)-2 isopropylaminoethanol.

For pharmaceutical purposes the particle size of the powder for the drug dispersion should desirably be no greater than 100 microns diameter, since larger particles may clog the valve or orifice of the container. Preferably the particle size should be less than 25 microns in diameter. Desirably the particle size of the finely-divided solid powder should for physiological reasons be less than 25 microns and preferably less than about 10 microns in diameter. The particle size of the powder for inhalation therapy should preferably be in the range 2 to 10 microns. There is no lower limit on particle size except that imposed by the use to which the aerosol produced is to be put.

The concentration of each medicament depends upon the desired dosage but is generally in the range 0.01 to 5% by weight.

The suspended drug should be insoluble in the aerosol formulation. Partial solubility of drug is undesirable since it can result in crystal growth due to Ostwald Ripening. The dissolved drug should be completely soluble in the aerosol formulation over the temperature range likely to be encountered by the product during storage and use.

The formulation of the invention may comprise an adjuvant e.g., to improve the solubility or stability of the drug to be dissolved. Suitable adjuvants are disclosed in EP-A-0372777 include hydrocarbons, dimethyl ether and alcohols. Ethanol is a preferred adjuvant and is generally present in an amount of from 2 to 20% by weight of the formulation.

The formulations may comprise surfactant. Suitable surfactants for aerosol formulations are well known and disclosed, for example, in EP0A-0372777. Preferred surfactants include oleic acid and sorbitan trioleate. Preferably the formulations are free of surfactant.

The propellant is preferably selected from P134a, P227 and mixtures thereof. Variation of the concentration of P134a and P227 in mixtures allows adjustment of the density of the aerosol formulation to match the density of the suspended drug. Density matching may decrease the rate of sedimentation or creaming of the suspending particles. Variation of concentration of any adjuvant may also contribute to density matching.

Suitable drugs which may be readily dissolved in the aerosol formulations include beclomethasone dipropionate and budesonide. These drugs may be completely dissolved in the formulations in therapeutic amounts e.g. 1 mg/ml in the presence of relatively low concentration of ethanol e.g. 8% by weight or less. Drugs such as flunisolide and butixocort propionate require higher therapeutic amounts e.g. 6 mg/ml and consequently may need larger amounts of ethanol to prepare a stable formulation.

Preferred drugs used in suspension include salbutamol base, salbutamol sulphate, terbutaline sulphate and pirbuterol acetate. A particularly preferred formulation comprises p134a and/or p227, salbutamol sulphate in suspension, beclomethasone dipropionate in solution and sufficient ethanol to maintain the latter in solution. The formulation is preferably free of surfactant.

Generally the salbutamol sulphate will be present in a concentration of from 2 to 5 mg/ml and beclomethasone dipropionate will be present in a concentration of from 1 to 5 mg/ml and the formulation will comprise from 4 to 15% by weight ethanol, preferably about 8% by weight ethanol.

The invention will now be illustrated with reference to the following Examples.

### EXAMPLES 1 TO 21

The formulations reported in the following Table 1 were prepared by addition to the beclomethasone dipropionate and salbutamol sulphate to a trimline can (a 100 ml container). The ethanol was then added, and homogenised using a Silverson mixer for five minutes. A non-metering valve was then crimped onto the can, and the propellant (p134a or p227) was added. The formulation was then chilled down in a cryobath and transferred after removing the valve to 10 ml aluminium vials and 10 ml PET bottles, which were sealed with suitable valves.

TABLE 1

| | Salbutamol | | | | | | |
|---|---|---|---|---|---|---|---|
| | sulphate | BDP mg/ml | Oleic mg/ml | Ethanol | P134a | P227 | Density |
| Example | mg/ml (% w/w) | (% w/w) | (% w/w) | % w/w | % w/w | % w/w | (g/ml) |
| 1 | 4.37 (0.385) | 3.93 (0.347) | 0.330 (0.029) | 14.5 | 85.5 | | 1.134 |
| 2 | 4.37 (0.385) | 3.93 (0.347) | | 14.5 | 85.5 | | 1.134 |
| 3 | 2.19 (0.193) | 1.96 (0.173) | 0.159 (0.014) | 14.5 | 85.5 | | 1.134 |
| 4 | 2.19 (0.193) | 1.96 (0.173) | | 14.5 | 85.5 | | 1.134 |
| 5 | 2.19 (0.187) | 1.98 (0.169) | 0.164 (0.014) | 8 | 92 | | 1.17 |
| 6 | 2.19 (0.187) | 1.98 (0.169) | | 8 | 92 | | 1.17 |
| 7 | 2.22 (0.164) | 2.0 (0.147) | 0.165 (0.012) | 5 | | 95 | 1.356 |
| 8 | 2.22 (0.164) | 2.0 (0.147) | | 5 | | 95 | 1.356 |
| 9 | 2.35 (0.208) | 1.96 (0.173) | | 15 | 85 | | 1.131 |
| 10 | 2.36 (0.206) | 1.96 (0.171) | | 12 | 88 | | 1.148 |
| 11 | 2.37 (0.204) | 1.97 (0.170) | | 10 | 90 | | 1.16 |
| 12 | 2.36 (0.202) | 1.98 (0.169) | | 8 | 92 | | 1.17 |
| 13 | 2.38 (0.201) | 1.98 (0.167) | | 6 | 94 | | 1.183 |
| 14 | 2.40 (0.177) | 2.0 (0.147) | | 5 | | 95 | 1.356 |
| 15 | 2.40 (0.183) | 2.0 (0.153) | | 10 | | 90 | 1.309 |
| 16 | 2.40 (0.194) | 2.0 (0.162) | | 10 | 45 | 45 | 1.235 |
| 17 | 2.40 (0.199) | 2.0 (0.166) | | 10 | 65 | 25 | 1.205 |
| 18 | 2.36 (0.202) | 0.98 (0.084) | | 8 | 92 | | 1.17 |
| 19 | 2.40 (0.207) | 1.0 (0.086) | | 10 | 90 | | 1.16 |
| 20 | 2.40 (0.177) | 1.0 (0.074) | | 5 | | 95 | 1.356 |
| 21 | 2.40 (0.194) | 1.0 (0.081) | | 10 | 45 | 45 | 1.235 |

The flocculation and sedimentation/creaming rates were assessed visually by means of time lapse photography.

### EXAMPLES 1 TO 8

Examples 1 and 2 flocculated at the quickest rate due to the larger concentrations of drug present.

Examples 7 and 8 formed floccules at the slowest rate since the formulations are substantially density matched. The flocculation rates of Examples 3 to 6 were similar, with Examples 5 and 6 flocculating slightly quicker than Examples 3 and 4.

Examples 1 to 8 all appeared as a white suspension; Examples 1 to 6 gradually sedimented and Examples 7 and 8 gradually creamed.

After standing for 3 days all formulations resuspended easily upon gentle agitation with no agglomerates or deposits sticking to the bottom of the vial.

### EXAMPLES 9 TO 13

All formulations sedimented. It was observed there was a trend in increasing rate of sedimentation as the ethanol concentration decreases.

### EXAMPLES 14

The drug flocculates and stays in suspension for about 1.5 minutes during which it begins to cream to the top. After 5 minutes larger floccules have formed which very slowly float to the top.

### EXAMPLES 15

The drug flocculates and stays in suspension for about 1 minute, after which large floccules begin to form. Sedimentation of the drug is very slow and has not started after 5 minutes. After 25 minutes very large floccules have formed, some of the drug is still in suspension with sedimentation starting to occur.

### EXAMPLES 16

The drug flocculates and begins to form large floccules as it simultaneously sediments. Sedimentation is apparent after 1 minute and clearly observed after 2 minutes.

### EXAMPLES 17

The drug flocculates and forms large floccules as it simultaneously sediments. Sedimentation begins after 30 seconds and is clearly observed after 1 minute. After 2 minutes nearly all the drug has settle to the bottom. The rate of flocculation and sedimentation of Example 17 is very similar and visually indistinguishable from that of Example 16.

### EXAMPLES 18

The drug flocculates and simultaneously sediments within a minute. After 2 minutes most of the drug has sedimented with a small amount still in suspension.

### EXAMPLES 19

The drug flocculates and simultaneously sediments after about 30 seconds. After 2 minutes most of the drug has sedimented. The sedimentation process was very similar to that of Example 18. The sedimentation rate for Example 19 which contains 10% EtOH, is slightly quicker than that of Example 18, which contains 8% EtOH. The density of Example 18 (1170.0 mg/ml) is more closely matched to that of Salbutamol sulphate than the density of Example 19 (1160.0 mg/ml).

### EXAMPLES 20

The drug flocculates after 30 seconds and creaming starts. After 1 minute floccules are becoming bigger and creaming is occurring slowly. After 3.5 minutes creaming is clearly observed but some of the drug is still in suspension. After 15 minutes most of the drug has creamed to the top The observations made are comparable and similar to those made on formulation Example 14 which has the same propellant system and density as Example 20. However, Example 14 (which has a greater BDP content of 2 mg/ml) takes slightly longer to cream, some of the drug still being in suspension after 30 minutes.

### EXAMPLES 21

The drug flocculates and simultaneously sediments after about 30 seconds. After 1 minute large floccules have formed and sedimentation is clearly taking place. After 2 minutes most of the drug has sedimented. The observations made are comparable and similar to those of formulation Example 16 which has the same propellant system and density. The sedimentation rate of Example 16 (BDP content 2 mg/ml) is quicker than that of Example 21 (BDP content of 1 mg/ml).

### EXAMPLES 22 to 26

The formulation reported in the following Table 2 were prepared.

TABLE 2

| | Example | | | |
|---|---|---|---|---|
| mg/ml | 22 | 23 | 24 | 25 |
| Salbutamol Sulphate | — | — | — | 2.40 |
| BDP | 2.00 | 2.00 | 1.00 | — |
| Terbutaline sulphate | 5.00 | — | — | — |
| Salbutamol Base | — | 2.00 | — | — |
| Pirbuterol Acetate | — | — | 6.30 | — |
| Budesonide | — | — | — | 1.00 |
| Ethanol (8%) | 93.040 | 93.280 | 93.016 | 93.328 |
| P134a (92%) | 1069.960 | 1072.720 | 1069.684 | 1073.272 |
| Total | 1170.00 | 1170.00 | 1170.00 | 1170.00 |

All the formulations were prepared using the method described previously with the exception of Example 25 which was homogenised using the Silverson mixer for the period of 10 minutes rather than the stated 5.

Each of the formulations were shaken in clear, plastic vials to give an even, white homogeneous suspension of drug.

### EXAMPLES 22

On shaking the drug flocculates and begins to form large floccules as it simultaneously sediments. Sedimentation is apparent after approximately 30 seconds and clearly observed at around 1 minute.

### EXAMPLES 23

On shaking the drug flocculates and begins to form large floccules and creaming begins. After 1 minute the floccules are becoming larger and are slowly rising to the surface. Creaming can be clearly observed after approximately 10 minutes. Complete creaming occurs after approximately 30 minutes.

### EXAMPLES 24

On shaking the drug flocculates and begins to form large floccules as it simultaneously sediments. Sedimentation is apparent after approximately 20 seconds and clearly observed at around 1 minute.

### EXAMPLES 25

On shaking the drug flocculates as it simultaneously sediments. Sedimentation is apparent after approximately 30 seconds and clearly observed at around 2 minutes. The vast majority of the suspended drug sediments within 3 minutes leaving a small quantity of drug still in suspension which eventually sediments after approximately 45 minutes.

1-15. (Cancelled).

16. A medicinal aerosol formulation comprising particles of a first drug suspended in a propellant and a second drug completely dissolved in the aerosol formulation, which additionally comprises an adjuvant selected from hydrocarbons, dimethylether and alcohols.

17. A medicinal aerosol formulation as claimed in claim 16 in which the propellant is free of CFC propellants.

18. A medicinal aerosol formulation as claimed in claim 17 in which the propellant comprises one or more hydrofluoroalkanes.

19. A medicinal aerosol formulation as claimed in claim 18 in which the propellant is selected from P134a, P227 and mixtures thereof.

20. A medicinal formulation as claimed in claim 16 which additionally comprises an adjuvant selected from hydrocarbons, dimethylether and alcohols.

21. A medicinal aerosol formulation as claimed in claim 16 in which the adjuvant is ethanol.

22. A medicinal aerosol formulation as claimed in claim 16 which comprises a surfactant.

23. A medicinal aerosol formulation as claimed in claim 22 in which the surfactant is selected from sorbitan trioleate and oleic acid.

24. A medicinal aerosol formulation as claimed in claim 16 which is free of surfactant.

25. A medicinal aerosol formulation as claimed in claim 16 in which the first drug is selected from salbutamol, terbutaline, pirbuterol and salts thereof.

26. A medicinal aerosol formulation as claimed in claim 16 in which the second drug is selected from beclomethasone dipropionate and budesonide.

27. A medicinal aerosol formulation as claimed in claim 16 in which the first drug is salbutamol or a salt thereof and the second drug is beclomethasone dipropionate.

28. A formulation as claimed in claim 27 comprising from 2 to 5 mg/ml salbutamol sulphate, 1 to 5 mg/ml ethanol and propellant selected from P134a, P227 and mixtures thereof.

29. A medicinal aerosol formulation as claimed in claim 28 which comprises ethanol in an amount of about 8% by weight of formulation.

30. A medicinal product comprising an aerosol container equipped with a dispensing valve, and containing an aerosol formulation as claimed in claim 16.

31. A pharmaceutical preparation for propellant driven metered dose inhalers having a fluorohydrocarbon as propellant, which comprises a combination of two or more active substances in a liquid phase wherein at least one active substance is present in dissolved form in the liquid phase by the use of one or more co-solvents other than the fluorohydrocarbon and at least one other active substance is in the form of suspended particles of the liquid phase.

32. Pharmaceutical preparation according to claim 31, wherein there are two active substances.

33. The pharmaceutical preparation according to claim 31, wherein the propellant is TG 134a, TG227 or combinations thereof.

34. The pharmaceutical preparation according to claim 33, wherein said one or more co-solvents comprises one or more pharmacologically tolerable alcohols, a pharmacologically tolerable ester, water or a mixture thereof.

35. The pharmaceutical preparation according to claim 33, wherein said one or more co-solvents is ethanol.

36. The pharmaceutical preparation according to claim 35 wherein the ethanol is present in a concentration of up to 25% by weight, based on the quantity of liquefied propellant.

37. The pharmaceutical preparation according to claim 36, wherein the ethanol is present in a concentration of up to 10% by weight, based on the quantity of liquefied propellant.

38. The pharmaceutical preparation according to claim 37, wherein the composition is stabilized by one or more stabilizers.

39. The pharmaceutical preparation according to claim 38, wherein said one or more stabilizer(s) is selected from one or more acids, salts and combinations thereof.

40. The pharmaceutical preparation according to claim 39, wherein the stabilizer(s) is hydrochloric acid, sulphuric acid, nitric acid, phosphoric acid, ascorbic acid, citric acid, benzalkonium chloride, ethylene diamine tetraacetic, their salts or combinations thereof.

41. The pharmaceutical preparation according to claim 40, wherein the stabilizer is citric acid.

42. The pharmaceutical preparation according to claim 41, wherein the stabilizer is present in an amount of up to 100 ppm.

43. The pharmaceutical preparation according to claim 42, wherein the stabilizer is present in an amount of up to 40 ppm.

44. The pharmaceutical preparation according to claim 40, wherein the preparation contains one or more surfactants or suspension-stabilizing agents.

45. The pharmaceutical preparation according to claim 44, wherein the one or more surfactants is selected from C₅₋₂₀ fatty alcohols, C₅₋₂₀ fatty acids, C₅₋₂₀ fatty acid esters, lecithin, glycerides, propyleneglycol esters, polyoxyethanes, polysorbates, sorbitan esters, carboyhydrates and combinations thereof.

46. The pharmaceutical preparation according to claim 44 wherein the one or more surfactants is selected from C₅₋₂₀ fatty acids, their esters and combinations thereof.

47. The pharmaceutical preparation according to claim 44 wherein one or more surfactants is selected from oleic acid, sorbitan mono-, di- or trioleate and combinations thereof.

48. The pharmaceutical preparation according to claim 44, wherein the one or more surfactants is oleic acid.

49. The pharmaceutical preparation according to claim 44 wherein the one or more surfactants or suspension-stabilizing agents is selected from a toxicologically acceptable polymer, block-polymer and combinations thereof.

50. The pharmaceutical preparation according to claim 31, wherein the active substance combination contains beclomethasone, budesonide, cromoglycinic acid, fenoterol, flunisolide, fluticasone, ipratropium, nedocromil, orciprenaline, oxitropium bromide, reproterol, salbutamol (albuterol), salmeterol, terbutalin, N-[[2,2-dimethyl-4-(2-oxo-2H-pyridin-1-yl)-5-trifluoromethyl-2H-1-benzopyran-3-yl]methyl]-N-hydroxy-acetamide, the esters, salts, solvates or combinations thereof.

51. The pharmaceutical preparation according to claim 50, wherein the active substance combination contains salbutamol sulphate (albuterol sulphate) and ipratropium bromide.
